(19) United States
(12) Patent Application Publication
(10) Pub. No.: US 2005/0019418 A1
(43) Pub. Date: Jan. 27, 2005

(54) Wart treatment

(76) Inventors: Charles E. Crutchfield, St. Paul, MN (US); Humberto Gallego, Minneapolis, MN (US)

Correspondence Address: BRIGGS AND MORGAN, P.A., 2200 IDS CENTER, MINNEAPOLIS, MN, 55402, US

(21) Appl. No.: 10/625,930

(22) Filed: Jul. 23, 2003

(51) Int. Cl. A61K 35/78; A61K 35/64; A61K 31/365; A61K 31/12

(52) U.S. Cl. 424/541; 424/773; 514/452; 514/679

(57) ABSTRACT

A single-use, delivery vehicle for the treatment of warts, consisting of a solution of cantharidin, acetone, and flexible collodion packaged in a crushable ampoule with an absorbent, applicator tip. A method of delivering wart-removing compositions to the skin, consisting of the steps of crushing an ampoule containing the wart-removing composition and pressing the absorbent tip against the wart.

### BACKGROUND OF THE INVENTION

The present invention relates to a single-use, delivery vehicle for treatment of cutaneous warts.

Cutaneous warts (verrucae) are benign, epithelial tumors characterized by the formation of thick, hyperkaratotic lesions. Human papillomavirus (HPV) is a double-stranded DNA virus, and is responsible for the appearance of warts. Virus particles reside in the basal layer of epithelia, but replicate only in the well-differentiated, superficial layer. The ensuing cellular proliferation gives rise to the characteristic morphology of warts. Several types of cutaneous warts have been described: common warts (verrucae vulgaris), plantar warts (verrucae plantaris), and flat warts (verrucae planus). Although verrucae are more commonly encountered in children and young adults, all ages may be affected. At particular risk are immunocompromised individuals. Humal papillomavirus may be transmitted indirectly through contact with the skin of an infected individual or by transmission of virus that has survived in warm, moist environments. The virus may also be transferred from one site to another when autoinoculation occurs upon traumatizing warts by scratching or biting. The incubation period is unknown, but may be several months or years.

Several treatment modalities for warts have been used in the past, but none have been universally successful. Chemotherapy, cryotherapy, surgery, immunotherapy, and other treatment methods have been attempted.

In the area of chemotherapy, cantharidin has been used for treatment of warts. Cantharidin is derived from the blister beetle (Coleopteres heteromeres) and poisons mitochondria, leading to acantholysis and vesicle formation. Plantar and periungual warts are commonly treated with cantharidin, typically as a 0.7% collodion solution after paring the wart with a blade.

Podophyllin may also be used as a chemotherapeutic agent. It is derived from the Podophyllum peltatum and p emodi plant species. It interrupts cellular activity at metaphase as it combines with tubulin. It also exerts its effect on verrucae by disrupting the microcirculation. Podophyllin is typically applied in a 25% liquid paraffin base or 0.5% podophyllotoxin in alcohol to the debrided wart surface. Cantharidin, alone or in combination with podophyllin, was commercially available until 1991 when the FDA ruled that salicylic acid could be obtained without a prescription, whereas other agents (cantharidin included) could not be marketed until safety and efficacy issues were resolved.

In typical application methods, cantharidin and podophyllin are dissolved in acetone and stored in single-use, multi-dose bottles. Accordingly, the composition has a tendency for the acetone to evaporate when the bottle is opened, leading to drying out. In addition, because of large amount of composition compared to the small amount needed per dose, practitioners have a tendency to use the same bottle for more than one patient. This can lead to unwanted cross-contamination. These application methods also do not provide precise control of delivery to the wart.

Single-use applicators are known for the delivery of isopropyl alcohol, tincture of iodine, and tincture of benzoin. However, such single-use applicators have not been used for the delivery of wart-removing compositions.

There is a need for an improved wart-removing composition and for an improved, single-use delivery vehicle.

### SUMMARY OF THE INVENTION

A single-use, delivery vehicle for the treatment of warts, consisting of a solution of cantharidin, acetone, and flexible collodion packaged in a crushable ampoule with an absorbent, applicator tip.

A method of delivering wart-removing compositions to the skin, consisting of the steps of crushing an ampoule containing the wart-removing composition and pressing the absorbent tip against the wart.

A principle object and advantage of the present invention is that it prevents the wart-removing composition from drying out.

Another principle object and advantage of the present invention is that cross-contamination between patients is prevented.

Another principle object and advantage of the present invention is that it minimizes waste.

Another principle object and advantage of the present invention is that it provide precise control of delivery of the wart-removing composition.

### BRIEF DESCRIPTION OF THE DRAWINGS

FIG. 1 is a schematic of the single-use, delivery vehicle of the present invention.

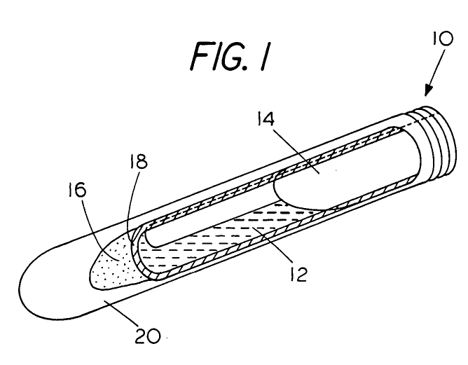

### DETAILED DESCRIPTION OF THE PREFERRED EMBODIMENT

The single-use, delivery vehicle for the treatment of cutaneous warts of the present invention is generally shown in the Figures as reference numeral 10.

The single-use, delivery vehicle 10 of the present invention comprises a solution 12 of cantharidin, in about 0.01% to about 20% by weight; acetone; and flexible collodion.

Preferably, the single-use, delivery vehicle 10 further comprises a crushable ampoule 14 in which the solution 12 is packaged. The single-use, delivery vehicle further preferably comprises an absorbent, applicator tip 16 separated from the crushable ampoule 14 by a wall 18. When the ampoule 14, is crushed, the wall 18 is disrupted, causing the solution 12 to flow to the applicator tip 16.

Preferably, the single-use, delivery vehicle 10 further comprises an outer wall 20 that protects the crushable ampoule 14.

The solution 12 may also further comprise podophyllin in about 1% to about 30% by weight.

The cantharidin may preferably be present in the solution 12 at about 0.7% by weight.

The solution 12 may also further comprise salicylic acid at about 2% to about 45% by weight.

The solution 12 may also further comprise tincture of benzoin.

To use the single-use, delivery vehicle 10, the user crushes the ampoule 14 disrupting the wall 18 and allowing the solution 12 to flow to the applicator tip 16. The applicator tip 16 is then pressed against the wart.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention.

1. A single-use, delivery vehicle for the treatment of cutaneous warts, comprising a solution of:
(a) cantharidin, in about 0.01% to about 20% by weight;
(b) acetone; and
(c) flexible collodion.

2. The single-use, delivery vehicle of claim 1, the solution further comprising podophyllin in about 1% to about 30% by weight.

3. The single-use, delivery vehicle of claim 1, wherein the cantharidin is present at about 0.7% by weight.

4. The single-use, delivery vehicle of claim 1, the solution further comprising salicylic acid at about 2% to about 45% by weight.

5. The single-use, delivery vehicle of claim 1, the solution further comprising tincture of benzoin.

6. The single-use, delivery vehicle of claim 1, further comprising a crushable ampoule with an absorbent, applicator tip.

7. A method of delivering wart-removing compositions to the skin, the composition being packaged in a single-use, crushable ampoule with an absorbent, applicator tip, comprising the steps of:
(a) crushing the ampoule, the ampoule containing a wart-removing composition; and
(b) pressing the absorbent tip of the ampoule against the wart.

8. The method of claim 7, where the wart-removing composition further comprises a solution of cantharidin, acetone, and flexible collodion.

9. The method of claim 8, wherein the solution further comprises about 0.01% to about 20% by weight cantharidin.

10. The method of claim 9, wherein the solution further comprises about 0.7% by weight cantharidin.

11. The method of claim 8, further comprising podophyllin.

12. The method of claim 11, wherein the solution further comprises about 1% to about 30% by weight podophyllin.

13. The method of claim 8, wherein the solution further comprises salicylic acid.

14. The method of claim 13, wherein the solution further comprises about 2% to about 45% by weight salicylic acid.

15. The method of claim 8, wherein the solution further comprises tincture of benzoin.
